(19) United States
(12) Patent Application Publication
(10) Pub. No.: US 2010/0030120 A1
(43) Pub. Date: Feb. 4, 2010

(54) Vibrotactile devices for controlled somato sensory stimulus during functionalmagnetic resonance imaging

(76) Inventors: Simon J. Graham, Toronto (CA); Donald B. Plewes, Toronto (CA); Wiliam E. McLlroy, Guelph (CA); W. Richard Staines, Toronto (CA)

Correspondence Address: DOWELL & DOWELL P.C., 103 Oronoco St., Suite 220, Alexandria, VA, 22314, US

(21) Appl. No.: 12/588,115

(22) Filed: Oct. 5, 2009

Related U.S. Application Data
(62) Division of application No. 10/126,329, filed on Apr. 19, 2002.
(60) Provisional application No. 60/284,917, filed on Apr. 20, 2001.

(51) Int. Cl. A61H 1/00; A61B 5/05

(52) U.S. Cl. 601/78; 600/411

(57) ABSTRACT

A functional magnetic resonance imaging (fMRI) compatible magnetomechanical vibrotactile device (MVD) uses wire coils having small oscillatory currents to interact with the large static magnetic field inherent to MRI scanners. The resulting Lorentz forces which are exerted on the MVD can be oriented to generate large vibrations that may be easily converted to translational motions as large as several centimetres. Representative data demonstrate the flexibility of MVDs to generate different well-controlled vibratory and tactile stimuli to activate special proprioceptive and cutaneous somatosensory afferent pathways.

### FIELD OF THE INVENTION

The present invention relates generally to functional magnetic resonance imaging equipment, and more particularly to an apparatus for providing somatosensory input.

### BACKGROUND OF THE INVENTION

Functional magnetic resonance imaging (fMRI) has fast become a widely used tool for investigating human brain function. Its prominence as an important tool is due largely to two underlying phenomena. First is the tight coupling of neuronal activation and hemodynamic/metabolic responses. Second is the presence of an endogenous contrast agent, paramagnetic deoxyhemoglobin, circulating through the circulatory system.

During an fMRI experiment, a subject is placed within the bore of a magnet and exposed to its magnetic field. The data are collected as a series of signal intensity measurements from small volume elements (“voxels”) that define the regions of interest or the whole brain. The resultant MRI signal of an image voxel is a sum of the water signals (or spins) from components such as tissue, blood or bulk cerebrospinal fluid (CSF). Because the signals are extremely small, fMRI procedures usually involve a time series of image acquisitions, which are recorded either in repeated blocks (e.g. 30 seconds repeated stimulation, 30 seconds rest) or as multiple discrete events spaced in time.

During the data acquisition period, sensory stimuli (inputs) for neural activation are presented to the subject. As neuronal activation is induced, the corresponding increased metabolic demands of the activated neurons are met by transient, focal increases in blood oxygenation, blood flow and blood volume. As a result, the deoxyhemoglobin content in the activated area decreases. Since deoxyhemoglobin in red blood cells is paramagnetic, when it is subjected to the magnetic field of the MRI scanner, there are susceptibility induced variations (local field distortions) in and around the surrounding tissue, blood and bulk CSF. The resultant MRI signal changes in response to these local field distortions. This deoxyhemoglobin effect has been called the blood oxygenation level dependent (BOLD) effect.

Functional MRI is an excellent experimental tool for probing the neural pathways associated with skin sensation—the somatosensory system. This system consists of neural pathways and associated receptors for tactile sensation (e.g., Pacinian corpuscles and Meissner's corpuscles), proprioception (sensation of the relative positions of body segments and of the body position in space), thermal sensation and nociception (pain perception). A central broad issue in human somatosensory system research is understanding, quantitatively, the factors that modulate somatosensory activation. This includes investigating the relationship between parameters associated with stimulus delivery, the specific peripheral receptors excited and the activation signals observed using fMRI. Numerous studies demonstrate the somatotopic organization of the primary somatosensory cortex. Somatotopic mapping at higher spatial resolution may ultimately have practical medical application in surgical planning (e.g., to resect tumours or epileptic foci) while minimizing somatosensory loss and paralysis. Other potential research applications include exploring the functional connectivity of the somatosensory cortex to other functionally connected brain regions and investigating impaired somatosensory function in disease states such as stroke.

To date, however, researchers have had limited success in realizing the full potential of fMRI as a somatosensory system research tool. Investigating the relationship between stimulus delivery, the peripheral receptors excited and the observed activation signals requires the capability to deliver well controlled, reproducible somatosensory stimuli that provoke robust neural activation when observed by fMRI. Previous attempts at careful somatosensory stimulus delivery (such as manual stimulation, pneumatic devices, electrical stimulation and piezoelectric devices) have met with limited success. Manual mechanical stimulation provokes robust neural activation when observed by fMRI, but depends completely on the ability of an experimenter to apply tactile stimuli consistently, which is extremely difficult. Pneumatic devices that use puffs of air suffer from this same limitation. Electrical stimulation of sensory nerves is more easily controlled, but it is not a natural central nervous system input and as such provides mixed results. Finally, while piezoelectric devices may be able to provide consistent tactile stimuli, they are incapable of producing large amplitude sensory stimuli necessary to provoke robust neural activation.

As such, there remains a strong need for an improved somatosensory input device which provides well controlled reproducible somatosensory input to the central nervous system, which is simple to operate and which is durable, easily adaptable, and relatively inexpensive to manufacture.

### BRIEF SUMMARY OF THE INVENTION

It is a benefit of the present invention to provide a magnetomechanical vibrotactile device (MVD) for providing well-controlled reproducible somatosensory input during functional magnetic resonance imaging.

It is therefore an object of the present invention to provide a MVD for generating somatosensory stimuli during functional magnetic resonance imaging, said MVD comprising a wire; a time-dependent current source coupled to each end of said wire; a former having an outer surface, said wire being wrapped around said outer surface in a plurality of coils.

In another aspect the invention provides a device for providing somatosensory stimuli during functional magnetic resonance imaging, said device comprising an array of magnetomechanical vibrotactile devices (MVDs), each MVD comprising a wire; a time-dependent current source coupled to each end of said wire; a former having an outer surface, said wire being wrapped around said outer surface in a plurality of coils.

In another aspect the invention provides a device for providing somatosensory stimuli during functional magnetic resonance imaging, said device comprising a series of aligned magnetomechanical vibrotactile devices (MVDs) fixed on to a coupling means, each MVD comprising a wire; a time-dependent current source coupled to each end of said wire; a former having an outer surface, said wire being wrapped around said outer surface in a plurality of coils.

In another aspect the invention provides a method of presenting somatosensory stimuli to a subject during functional magnetic resonance imaging (fMRI), said method comprising the steps of providing a coil of wire around a former; applying current to the coil; placing the coil within the magnetic field of a fMRI scanner; and, applying the resulting oscillations generated by the coil to the skin of the subject to stimulate the somatosensory receptors of the subject.

Further objects and advantages of the invention will appear from the following description, taken together with the accompanying drawings.

### BRIEF DESCRIPTION OF THE DRAWINGS

In the accompanying drawings:

FIG. 1 is a perspective view of a magnetomechanical vibrotactile device (MVD), according to the present invention;

FIG. 2A is a perspective view of the MVD of FIG. 1 and the magnetic moment that results when a clockwise current is applied;

FIG. 2B is a perspective view of the MVD of FIG. 1 and the magnetic moment that results when a counter-clockwise current is applied;

FIG. 3A is a perspective view of the MVD of FIG. 2A when the longitudinal axis of the MVD is approximately orthogonal with the external magnetic field Bₒ;

FIG. 3B is a perspective view of the MVD of FIG. 2B when the longitudinal axis of the MVD is approximately orthogonal with the external magnetic field Bₒ;

FIG. 4A is a schematic drawing of the Lorentz forces which are generated during operation of the MVD of FIG. 1 when the longitudinal axis of the MVD is parallel with the external magnetic field Bₒ;

FIG. 4B is a schematic drawing of the forces which are generated during operation of the MVD of FIG. 1 when the longitudinal axis of the MVD is orthogonal with the external magnetic field Bₒ;

FIG. 5A is a perspective view of the MVD device of FIG. 1 coupled to a brush to produce a tactile brushing stimulus;

FIG. 5B is a perspective view of the MVD device of FIG. 1 coupled to a footpiece with a velcro strip to produce a tactile tapping stimulus;

FIG. 6A is a front view of an array of MVD devices aligned and fixed on a coupler;

FIG. 6B is a side view of an MVD of FIG. 6A fixed to the coupler;

FIG. 6C is a front view of an alternate array of MVDs devices aligned and fixed on a coupler;

FIG. 6D is a side view of the MVD devices of FIG. 6C fixed to the coupler;

FIG. 7 is a perspective view of an array of the MVD devices of FIG. 1 positioned on a subject's hand;

FIG. 8 is a front view of another embodiment of a MVD according to the present invention consisting of a spherical former and three sets of wire coils, each set of wire coils being oriented orthogonal to the others;

FIG. 9 is a block diagram illustrating one implementation of two MVD devices of FIG. 1 within functional magnetic resonance imaging (fMRI) equipment;

FIG. 10A is a top partial cutaway view of another embodiment of a MVD according to the present invention consisting of two coupled coils and an actuator assembly that transfers vibrational motion into vertical displacement;

FIG. 10B is a top surface view of the MVD of FIG. 10A showing the mounted footpiece and Velcro strip for producing tactile tapping stimuli;

FIG. 11A is a graph showing time series data for a voxel in primary somatosensory cortex (SI) showing activation produced by manual brushing;

FIG. 11B is a graph showing time series data for a voxel in primary somatosensory cortex (SI) showing activation produced by automated brushing using the MVD of FIG. 10 adapted to work in the mode illustrated in FIG. 5A;

FIG. 12A is an activation map showing activation in the primary somatosensory cortex (SI) produced by placement of the MVD of FIG. 10 over the thenar muscles of the hand;

FIG. 12B is an activation map showing activation in the secondary somatosensory cortex (SII) produced by placement of the MVD of FIG. 10 over the thenar muscles of the hand;

FIGS. 13A, 13B, and 13C show activation in the primary somatosensory cortex (SI) produced by driving the MVD of FIG. 10 with inputs of 50 mV, 150 mV and 250 mV, respectively; and

FIGS. 14A, 14B, and 14C show proprioceptive activation of the primary somatosensory cortex (SI), secondary somatosensory cortex (SII), supplementary motor area (SMA) and parietal association cortex (Brodman Area 7) produced by placement of the MVD of FIG. 10 over the patellar tendon.

### DETAILED DESCRIPTION OF PREFERRED EMBODIMENTS

### Single MVD

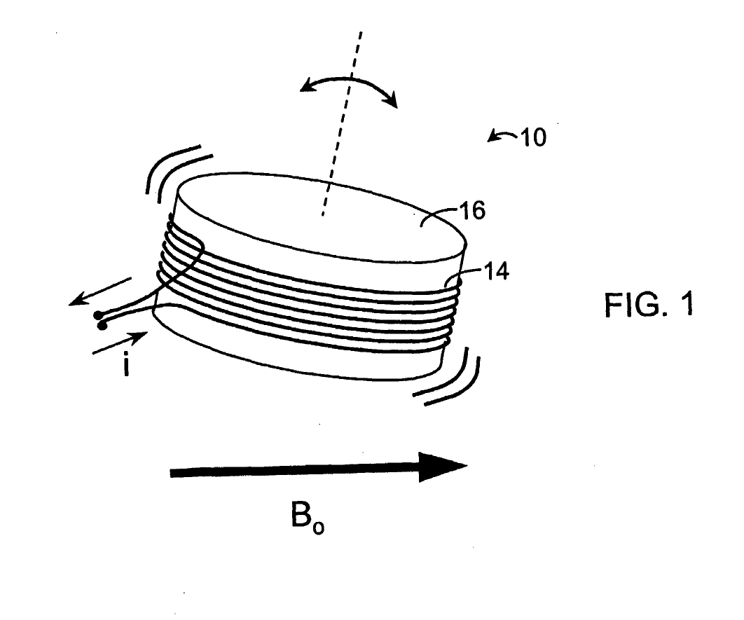

Reference is first made to FIG. 1, which shows a magnetomechancial vibrotactile device (MVD) 10 made in accordance with a preferred embodiment of the invention. MVD 10 is adapted to provide a well-controlled, reproducible somatosensory stimulus by using the static magnetic field Bₒ of a magnetic resonance imaging (MRI) scanner.

MVD 10 includes a wire coil 14 wrapped around a former 16 constructed from MRI-compatible material, such as plastic. Wire coil 14 consists of loops of fine wire through which a small current i is driven (as shown in FIG. 1). In an alternate embodiment of the invention, wire coil 14 is embedded in former 16 and coiled within the outer surface of the former 16. In a further embodiment, wire coil 14 is coiled below an inner surface 17 (not shown) of former 16 in a plurality of coils.

### MVD Outside the Static Magnetic Field of an MRI Scanner:

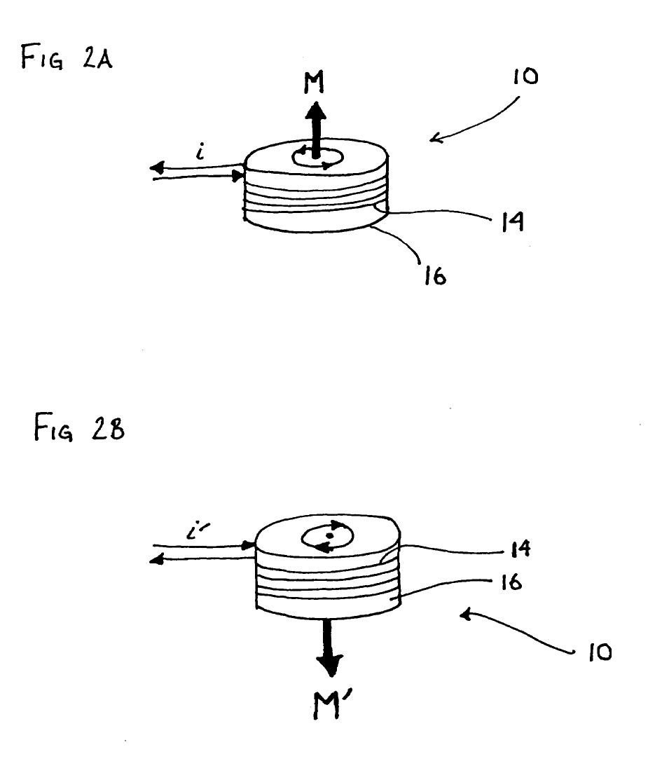

Reference is made to FIGS. 2A-2B, which show the direction of the magnetic moment that results when a current i is driven through wire coil 14. Referring to FIG. 2A, when small current i is driven through wire coil 14. One end of the current source is fixed to one end of the coil and the other end of the current source to the other end of the coil. A magnetic moment M (and a corresponding small magnetic field) is generated. The direction of magnetic moment M (and the corresponding field) is orthogonal to the direction of current i and may be determined by the “right hand rule”, which states that if we curl the fingers of the right hand in the direction of the current flow around the loop, the thumb points in the direction of the magnetic moment of the current loop.

Referring to FIG. 2B, if the direction of current i is reversed (it, the polarity of the resultant magnetic moment M′ will still be orthogonal to the flow of current i′, but will point in the direction opposite to the original magnetic moment M.

Thus, if an alternating current is driven through wire coil 14, the polarity of the resultant magnetic moment will alternate accordingly. The rate at which the polarity of the resultant magnetic moment alternates varies as a function of the frequency of the alternating current.

### MVD Inside the Static Magnetic Field of an MRI Scanner:

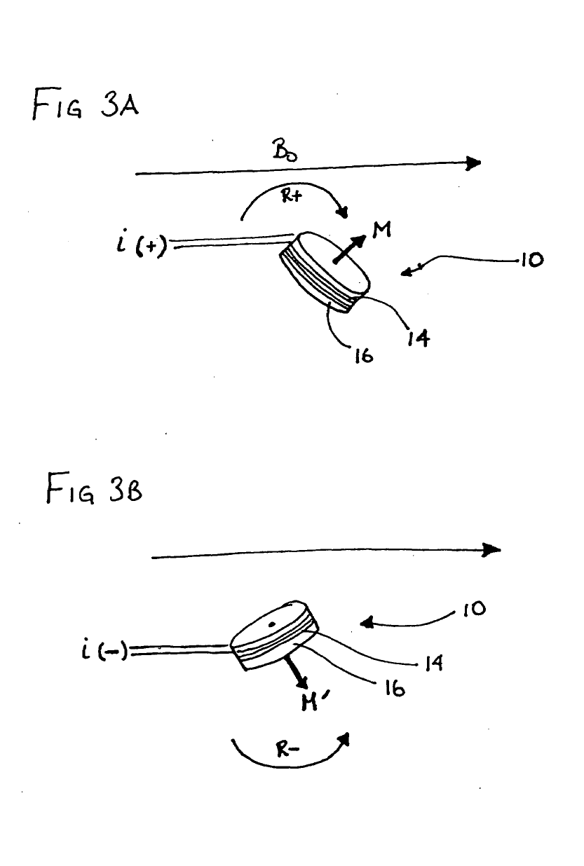

Reference is made to FIGS. 3A-3D which show the action of MVD 10 in the presence of an external magnetic field Bₒ. In the presence of a strong external magnetic field, Bₒ, the magnetic moment M (and thus the coil and former) experiences a turning force, or torque T, tending to align the magnetic moment with the external field, Bₒ. Whether the coil will tend to align or anti-align with the external filed, Bₒ, depends on the polarity of the applied current. FIG. 3A shows a depiction where a current flowing in the positive direction i(+) is driven through wire coil 14, resulting in a net clockwise rotation, R+. FIG. 3B shows a depiction where a current flowing in the negative direction i(−) is driven through wire coil 14, resulting in a net counter-clockwise rotation, R−.

The physical principle behind the operation of MVD 10 when placed within an external magnetic field is the Lorentz force law provided by the vectorial equation:

F=qv×B (1)

where F is the force produced on a charge q moving with velocity v in a region with uniform magnetic flux density B. Therefore, when a time-dependent current, i (t) is applied to the circumferentially-wound coil, in the magnetic field Bₒ of a MRI scanner, Lorentz forces are generated mutually perpendicular to the vectors of the current flow and the magnetic field. The force magnitude at each point in the wire of wire coil 14 is then:

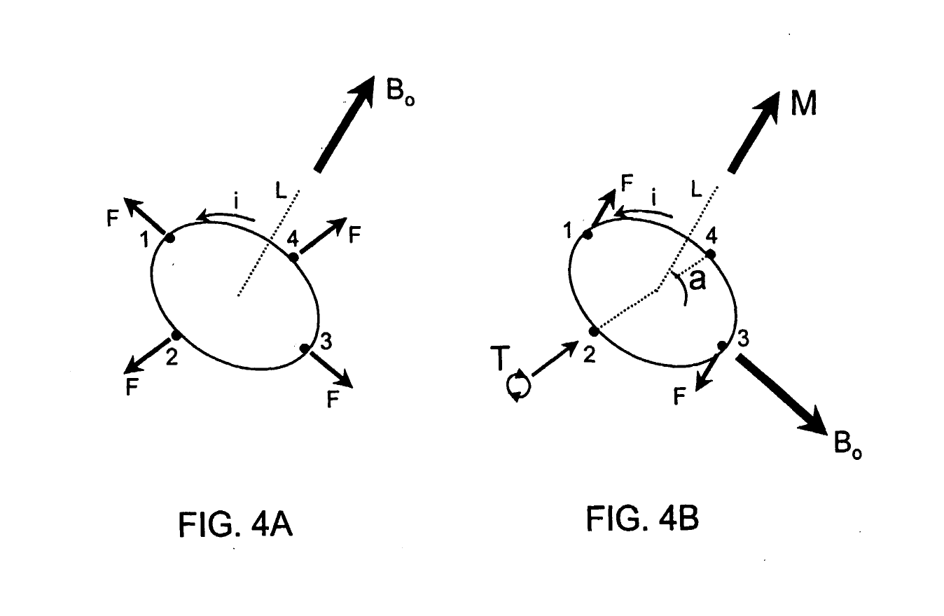

F(x,y,z,t)=2πri(x,y,z,t)Bₒ (2)

where r is the radius of the coils in wire coil 14. These forces are illustrated schematically in FIGS. 4A and 4B for two different single-loop coil orientations.

FIG. 4A shows the forces generated at points 1, 2, 3, and 4 during operation of MVD 10 in the case where the longitudinal axis L (dotted line in FIG. 4A) of the wire coil 14 is parallel to the external magnetic field Bₒ. FIG. 4B shows the forces generated at the same points when the longitudinal axis L (dotted line in FIG. 4B) of the wire coil 14 is orthogonal to the external magnetic field Bₒ. The forces produced when the longitudinal axis of the wire coil 14 is parallel to the external magnetic field Bₒ (i.e. as shown in FIG. 4A) are tensile at points 1, 2, 3, and 4. In contrast, when the longitudinal axis of the wire coil 14 is orthogonal to the external magnetic field Bₒ(i.e. as shown in FIG. 4B), the direction of force F at point 1 is opposite to the direction of force F at point 3, and a torque effect T is produced about the axis through points 2 and 4, as described by:

T=M×Bₒ (3)

where M is the magnetic moment of the coil (iπr²) pointing in the direction of the longitudinal axis L of wire coil 14 and which depends (in time) on the angle a between the longitudinal axis L of wire coil 14 and the external magnetic field Bₒ.

This torque T can then be used to actuate mechanically the motion of MVD 10. In particular, if the current i flowing through wire coil 14 is oscillatory, wire coil 14 can be used to generate a reproducible, well-controlled vibratory or tactile stimulus. Since the external magnetic field Bₒ is so strong in MRI scanners, very little driving current is required. For example, using equation (3) above, the force equivalent to the weight of 1 gram can be achieved at magnetic field strength of 1.5 Tesla by driving a current of 70 mA through a coil having a 3 cm radius. In stronger fields, even less current is required because for a constant torque, current is inversely proportional to magnetic field strength. Therefore, MVDs 10 ranging in radius from several centimetres to fractions of a centimetre are feasible.

MVDs 10 can be implemented in a variety of different configurations and applications. In its simplest application, the MVD 10 stimulus is produced by the coil and former of FIG. 1 and can be applied topically at arbitrary anatomical locations, to elicit varied stimulus responses to vibration in the subject. Different surface coatings on MVD 10 can be used to stimulate different sensory pathways (e.g. rough or pin cushion surface coatings can be used to provoke a pain response).

### Transductive Components:

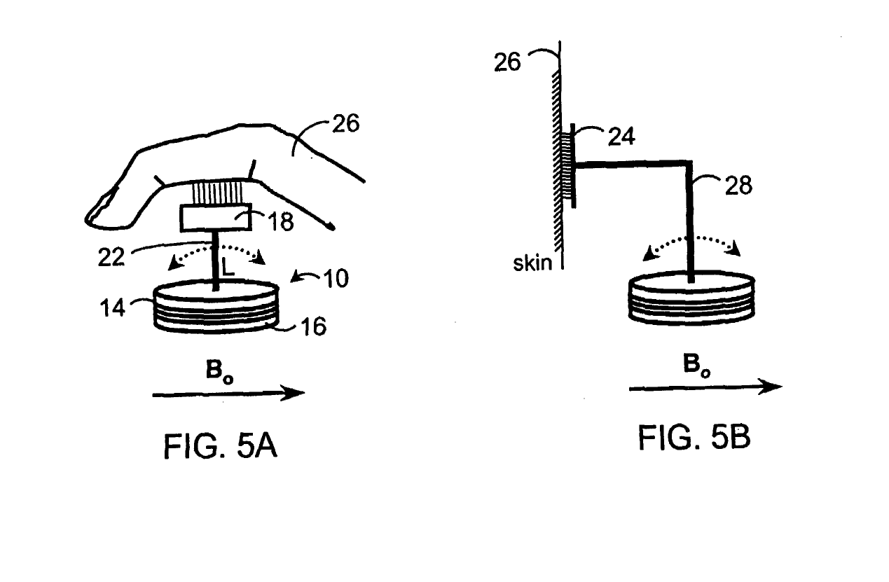

FIGS. 5A and 5B illustrate how the vibratory motion of MVD 10 can be converted to brushing motion (FIG. 5A) and tapping motion (FIG. 5B) for application to the skin 26 of a subject. In both cases, the vibratory motion of MVD 10 has been coupled with transductive components 18 and 24 to change the nature of the stimulus. Specifically, in FIG. 5A, transductive component 18 (e.g. a plastic brush) is coupled to MVD 10 through a straight rigid connector 22 oriented along the longitudinal axis L of MVD 10. Since the longitudinal axis L is orthogonal to the external magnetic field Bₒ, torque T acts on MVD 10 as depicted in FIG. 4B (as shown by the dotted line in FIG. 5A). Accordingly, transductive component 18 produces a side-to-side brushing motion against skin 26. In contrast, in FIG. 5B, transductive component 24 (e.g. a footpiece with a velcro strip) is coupled to MVD 10 through a L-shaped rigid connector 28 which converts the torque T acting on MVD 10 into a side-to-side motion (as shown by the dotted line in FIG. 5B) which provides a tapping motion to skin 26 oriented as shown.

These stimuli (vibration, brushing, and tapping) are particularly important in practise because they are very potent and elicit strong neural activation. When applied to tendons, tapping stimuli will recruit muscle spindle receptors in the associated muscle which are extremely sensitive to muscle stretch and which play a major role in proprioception, enabling examination of body and limb position awareness. Depending on the stimulation strength, muscle vibration may evoke an illusory sensation of motion which can be used to investigate sensory conflict. Other possibilities include vibration of the eyeballs to observe the effect of phosphenes on visual input, auditory stimulus by vibration on the skull, and even internal stimulation through various body cavities or by surgical incision. Such MVDs can also be used as prompts to control subject responses to specific stimuli during interactive fMRI experiments involving other cognitive tasks.

### Multiple MVDs (Aligned):

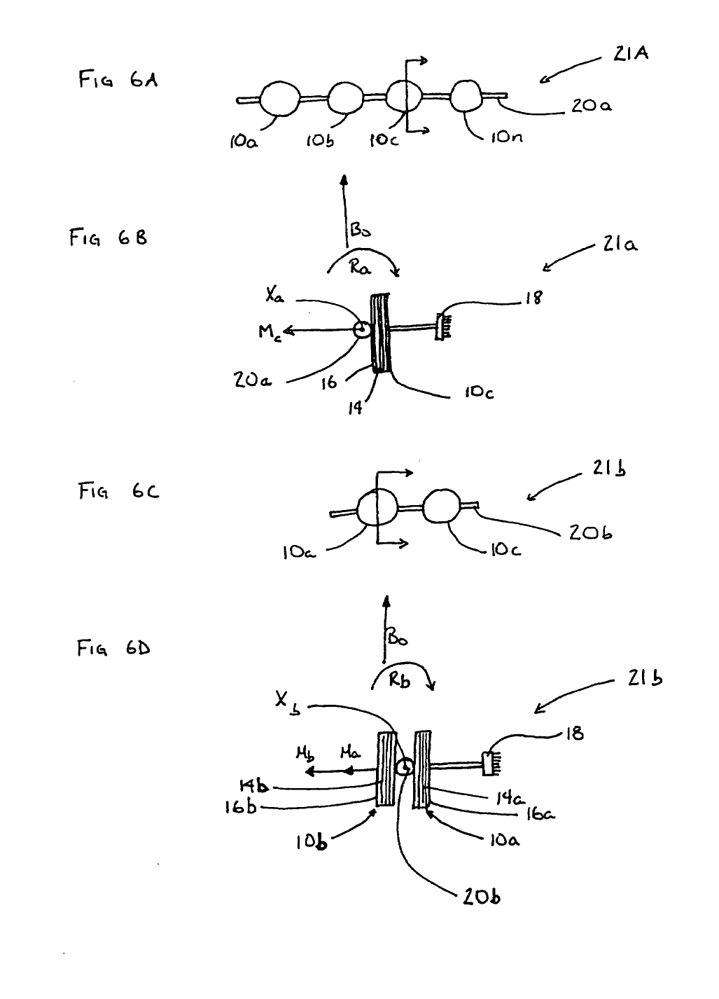

The resultant torque T produced by MVD 10 increases as a function of the amplitude at which current i is driven through wire coil 14. If, however, the current i is held constant, then employing multiple MVDs, which act in concert and simultaneously, also increases the resultant torque T as the number of MVDs used simultaneously increases. FIG. 6A-6D shows the exemplified use of multiple MVDs 10a, 10b, 10c and 10n, aligned and fixed to a coupler 20 to create a MVD-coupler unit 21, thus permitting the simultaneous action of the multiple aligned MVDs. FIG. 6A shows one embodiment of the invention where MVDs 10a, 10b, 10c and 10n are aligned adjacent to each other and on one side of the coupler 20a. MVDs 10a, 10b, 10c and 10n may be fixed to coupler 20a with an adhesive, a nail or screw made of MRI compatible material or any other affixing means, which are compatible for use in an MRI scanner, known to those skilled in the art. The former 16 of MVD 10 may also be embedded in the coupler. Additionally, the former 16 of MVD 10 may also be manufactured at the same time as coupler 21a as a single unit.

FIG. 6B shows a cross-section through MVD 10c of the MVD-coupler unit 21a of FIG. 6A. A transductive component 18 is attached. When MVD-coupler unit 21a is placed within a magnetic field Bₒ and a current i is driven through wire coils 14a (not shown), 14b (not shown), 14c and 14n (not shown), the resultant magnetic moments Ma (not shown), Mb (not shown) Mc and Mn (not shown) are generated, the total magnitude of which increases as the number of MVDs used increases. Thus, the MVD-coupler unit 21a experiences a net torque T tending to align the magnetic moments with the external field Bₒ. The MVD-coupler unit 21a rotates about the longitudinal axis Xa of coupler 20a in the direction represented by arrow Ra.

FIG. 6C shows another embodiment of the invention where the MVDs 10a, 10b, 10c and 10n are aligned and also paired on opposite sides of a coupler 21b to create MVD-coupler unit 21b. The MVDs 10a, 10b, 10c and 10n may be fixed to coupler 21b in the same manner as the MVD-coupler unit 21a of FIG. 6A.

FIG. 6D shows a cross-section view through coupler 20b and MVDs 10a and 10b of MVD-coupler unit 21b of FIG. 6C. A transductive component 18 is attached. When MVD-coupler unit 21b is placed within a magnetic field Bₒ and a current i is driven through wire-coils 14a, 14b, 14c (not shown) and 14n (not shown), the magnetic moments of MVDs 10a, 10b, 10c (not shown) and 10n (not shown) (Ma, Mb, Mc (not shown) and Mn (not shown)) (and thus the MVD-coupler unit 21b) experiences a net torque T (the total magnitude of which increases as the number of MVDs used increases) tending to align the magnetic moments with the external field Bₒ. MVD-coupler unit 21b rotates about the longitudinal axis Xb of the coupler 20b in the direction represented by arrow Rb.

### Multiple MVDs (Array):

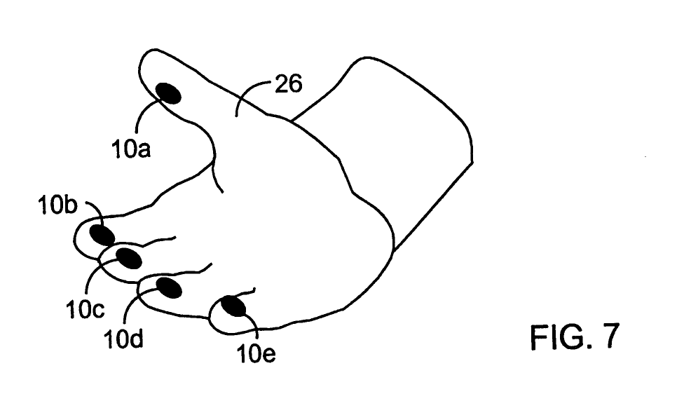

FIG. 7 shows the use of multiple MVDs 10a, 10b, 10c, 10d, and 10e, in combination on the skin 26 of a subject's hand. Generation of coordinated or successive stimuli using MVDs 10a, 10b, 10c, 10d, and 10e at multiple anatomical sites provides improved capability to investigate left-right asymmetries, somatotopic organization and inter- and intra-hemispheric neural pathways. MVDs 10a, 10b, 10c, 10d, and 10e could also be used at one anatomical location (not shown) to generate asymmetric streams or volleys of sensory input, for the purpose of simulating the physiological sensory discharge patterns such as those produced by walking or some other type of physical exercise. For such applications, it is contemplated that various types of apparel (not shown) could be utilized for the purpose of affixing MVDs to different body regions (e.g. the array shown in FIG. 7 would include a glove (not shown) containing one MVD for each finger). It is also contemplated that a linear array of MVDs 10a, 10b, 10c, 10d, and 10e could be mounted within a handgrip (not shown). In this way, use of an array of MVDs can be used to research the biophysics of fMRI and associated hemodynamic response produced by vibratory stimuli, to investigate linearity, superposition, and temporal response of activation.

### Spherical MVD:

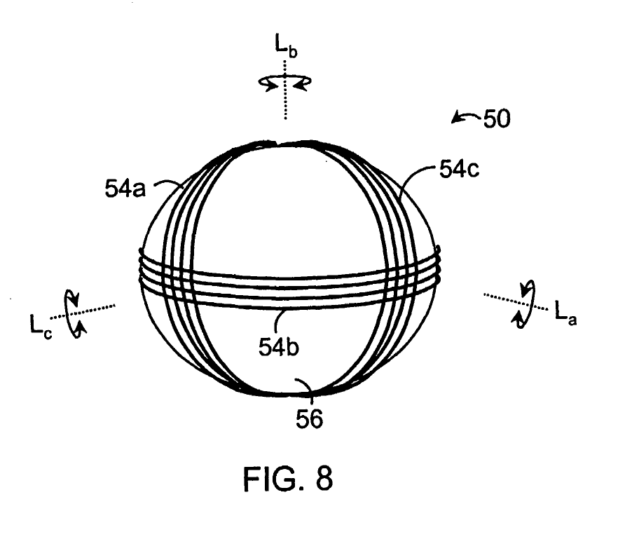

FIG. 8 illustrates a further embodiment of the present invention, designated generally as MVD 50 for providing improved stimulus accuracy and reproducibility. Specifically, MVD 50 uses a spherical former 56 which supports three wire coils 54a, 54b, and 54c which are wrapped around former 56, such that each wire coil has a longitudinal axis Lₐ, L_{b}, L_{c} which is mutually perpendicular to the longitudinal axis of the two other wire coils. The currents provided to individual wire coils 54a, 54b, and 54c can be tuned to achieve constant vibration in an arbitrary orientation, depending on where and how MVD 50 is positioned and oriented, respectively. The tuning procedure is facilitated by the fact that the time varying magnetic field produced by one wire coil will affect the magnetic fields associated with the other two coils. This configuration is particularly advantageous in practice since MVDs are likely to be used for investigations of the hands and lower limbs in the scanner's fringe field. The fringe field is more spatially inhomogeneous than the interior of the scanner where fMRI is performed, and could lead to degraded stimulus delivery.

To calibrate vibrotactile stimuli directly, MVD 50 can be used with MR elastography, which provides accurate measurements of tissue displacement at the skin surface. Elastographic measurements, which are capable of measuring submicron displacements at physiologically relevant vibration frequencies, can be used in a feedback loop (as conventionally known) to ensure consistent and reproducible displacement over multiple fMRI examinations and subject populations.

### Implementation:

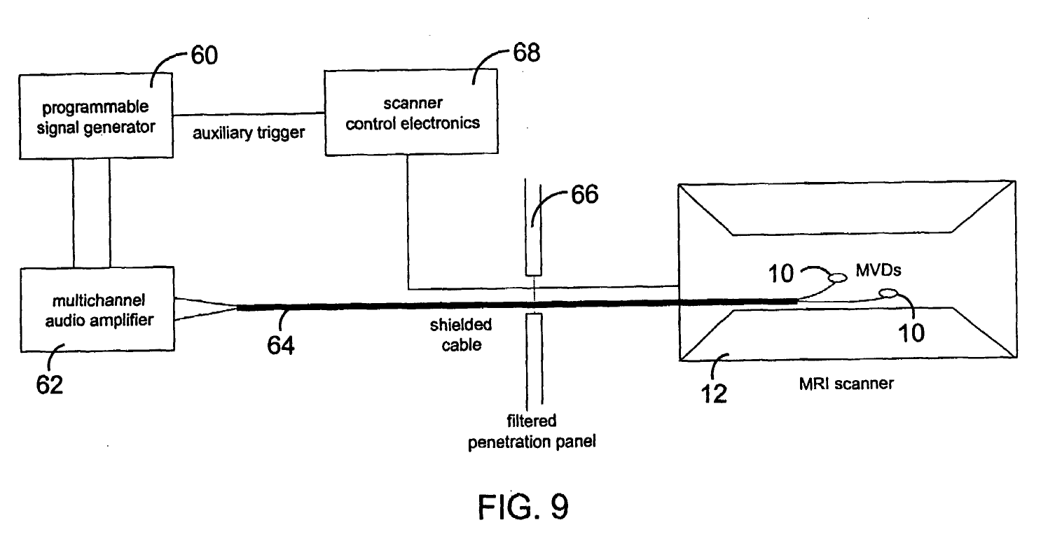

FIG. 9 schematically illustrates a basic implementation of two MVDs 10 in association with MRI scanner 12. The driving circuit for each MVD 10 consists of a programmable signal generator 60 coupled to a multichannel audio amplifier 62, from which driving currents are transmitted by non-ferromagnetic, shielded cable 64 through the filtered penetration panel 66 of the scanner room to the MVDs 10. The combination of shielded cable 64 and filtered penetration panel 66 ensure that MVDs 10 do not degrade the signal-o-noise ratio of fMRI signals. The signal generator is attached to the auxiliary trigger of the scanner control electronics 68 to ensure that stimuli are synchronized in time with fMRI acquisition.

The stimulus provided by MVDs 10 depends on the input current signal from the signal generator. It is possible to provide a standard alternating current (AC) signal with fixed amplitude and frequency. However, amplitude and frequency modulated signals are also possible, as well as random stimulus presentation. These variations would allow investigation of the sensitivities of different subcutaneous sensory receptors, and would allow investigation of receptor properties for cognitive tasks that require attention to the nature of the stimulus. It is contemplated that the characteristics of these input signals can also be varied to reduce habituation to the stimulus.

The torque and vibrational frequency of the stimulus presented by MVD 10 are two variables particularly important to the quality of mechanoreceptor stimulation. The torque exerted by MVD 10 varies as a function of the current, i, driven through wire coil 14 and also as a function of the number of MVDs that act in concert. As current, i, increases, the resultant torque exerted by MVD 10 increases accordingly and as a result, the intensity or amplitude of the stimulus presented by MVD 10. If the current, i, is not varied, but rather multiple MVDs are aligned to operate simultaneously and in concert, the resultant torque exerted by the multiple MVDs increases as a function of the number the MVDs used.

The vibrational frequency at which MVD 10 operates varies as a function of the frequency of the alternating current, i, that is driven through wire coil 14. As the frequency of the alternating current, i, increases, the vibrational frequency of MVD 10 increases accordingly and as a result, the frequency of the vibrational stimulus presented by MVD 10 increases.

The ability to vary the characteristics of the stimulus presented by MVD 10 by altering the current, i, and the number of MVDs working in concert is particularly relevant when one considers that the different mechanoreceptors that are stimulated with MVD 10 during an fMRI experiment each respond preferentially to a stimulus with a particular set of characteristics. For example, Meissner's corpuscles respond optimally to stimulus vibrations in the 20-50 Hz range, while Pacinian corpuscles respond optimally to vibratory stimuli in the 100-300 Hz range. As such, the characteristics of MVD 10 may be altered to preferentially recruit a specific class of mechanoreceptors.

### Experimental Test Results

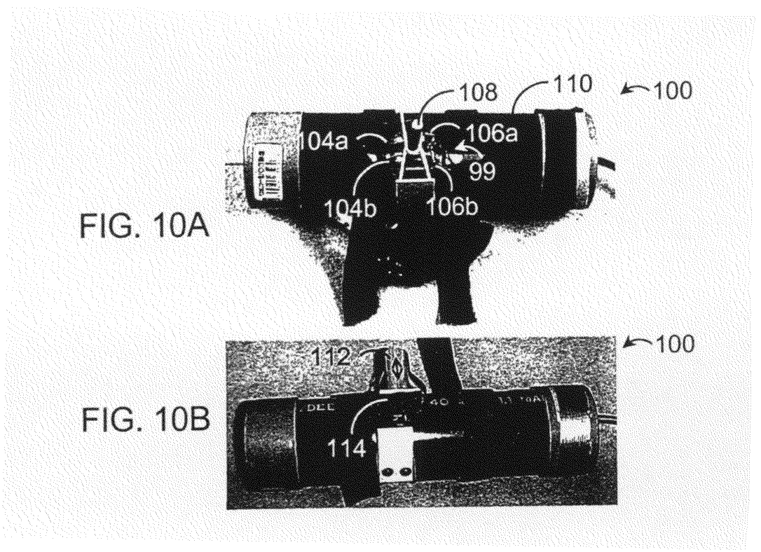

FIGS. 10A and 10B illustrate an experimental MVD prototype 100 that was designed preferentially to enhance the reproducibility of the stimulus and to allow the somatosensory content of the stimulus to be altered flexibly from simple vibration to that shown in FIGS. 5A and 5B. As shown through the cutaway portion 99 in FIG. 10A, MVD prototype 100 includes a pair of identical wire coils 104a and 104b wound around two identical formers 106a and 106b, respectively. The wire coils 104a and 104b are aligned such that the torque is produced by MVD 100 is double what it would be using only one wire coil 104a or 104b. Each wire coil 104a and 104b have a nominal 4 cm diameter and consist of speaker wire. Wire coils 104a and 104b are fixed in place to a central wooden doweling rod 108 and housed within PVC pipe 110.

FIG. 10B illustrates how a subject's finger is provided with a tactile tapping stimulus (as detailed in FIG. 5B). Specifically, a shaft is attached to MVD 100 containing a footpiece 112 with a plastic velcro strip 114 that undergoes translational motion (indicated by arrows). Alternatively, the prototype is easily reconfigured to produce a brushing stimulus by attaching a velcro strip 114 directly to central rod 108 within a slotted opening to rub the glabrous (palmar) surface of the finger (shown schematically in FIG. 5A).

Now referring to FIG. 9, MVD 100 is implemented within the overall experimental system using commercially available electronic equipment. Specifically, programmable signal generator 60 can be implemented using a function generator (e.g. Tektronix #AFG 5102 manufactured by Tektronix of Oregon) or the signal generation package in Labview coupled with a digital-to-analog converter (manufactured by National Instruments, Inc. of Virginia) housed in a personal computer. This equipment produces a driving voltage (nominally 50-100 mV) to multichannel audio amplifier 62. The driving signal is amplified using a commercially available audio amplifier (e.g. Bryston #3B-ST manufactured by Bryston of Ontario) passed through a suitable shielded cable 64 (e.g. non-ferromagnetic RG48 coaxial shielded cable) and routed at the penetration panel 66 to the fMRI scanner 12 using a filtered 9-pin D-sub connector.

Functional MRI experiments were conducted using an MRI scanner 12 operating at 1.5 T (e.g. Signa manufactured by General Electric Medical Systems of Wisconsin using a NV/i platform and software version LX 8.2.5). Functional imaging was performed using single-shot gradient echo spiral k-space acquisition with offline gridding, magnetic field inhomogeneity correction, and image reconstruction. Nine slices 7 mm thick were acquired axially from the most superior aspect of the brain inferiorly (field of view FOV=20 cm, TE/TR/=40/1500/70°). Anatomical axial images with high spatial resolution were performed using spoiled gradient echo imaging (FOV=22 by 16.5 cm, TE/TR/=5.4112.4/35°, 256 by 192 acquisition matrix, 124 slices 1.4 mm thick) to localize regions of somatosensory activation. Stimuli were presented in blocked format (30 s on/30 s off, 5 cycles). Activation maps were created using the AFNI software package with boxcar correlation and a confidence level of 95% (Bonferoni corrected), including image coregistration in three spatial dimensions to reduce the effects of small head motions.

Prior to applying MVDs 10 on human subjects, experiments were performed placing the MVD 10 designed to provide vibratory stimulation on the surface of a quality assurance phantom. Axial echo planar images were acquired in the plane of the MVD 10 both in the absence and the presence of typical driving current (100 mV at 100 Hz). Region of interest measurements were then performed to assess the noise present in magnetic resonance images acquired with high receiver bandwidth when MVDs 10 were strongly driven. Following these experiments, representative data were acquired using MVDs 10 in young healthy adults (within an age range of 25 to 30 years). The prototype MVD 100 was operated at nominally 1 Hz in brushing mode, and 20 Hz in tapping mode. In all cases, subjects were confined within the standard quadrature birdcage head coil using a vacuum pillow (e.g. manufactured by Vac Fix Systems Inc. of Denmark). Head displacements were less than 1 mm based on image coregistration of time series data.

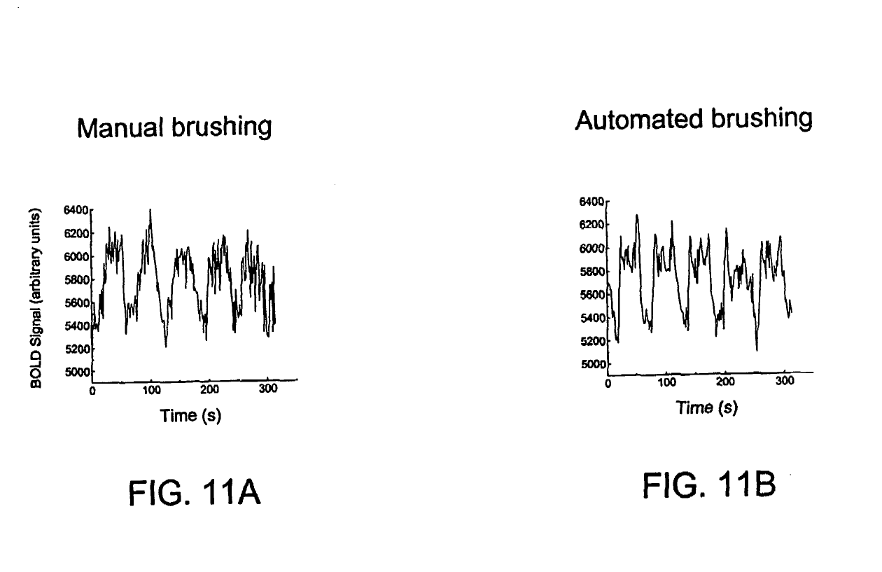

FIGS. 11A and 11B show representative time series data from an image voxel in primary somatosensory cortex (SI) resulting from tactile stimulation of the finger manually at approximately 1 Hz using a toothbrush, and automatically using the prototype MVD 100, respectively. The brushing force was similar in both cases. Both stimuli produced robust activations, although the time series produced using the MVD 100 (i.e. FIG. 11B) clearly exhibits more consistent, reproducible activation. The capability to automate the output of the MVD 100 in a more reproducible manner than is achievable with manual stimulation, reduces variability in time series data and increases the sensitivity for mapping brain activation.

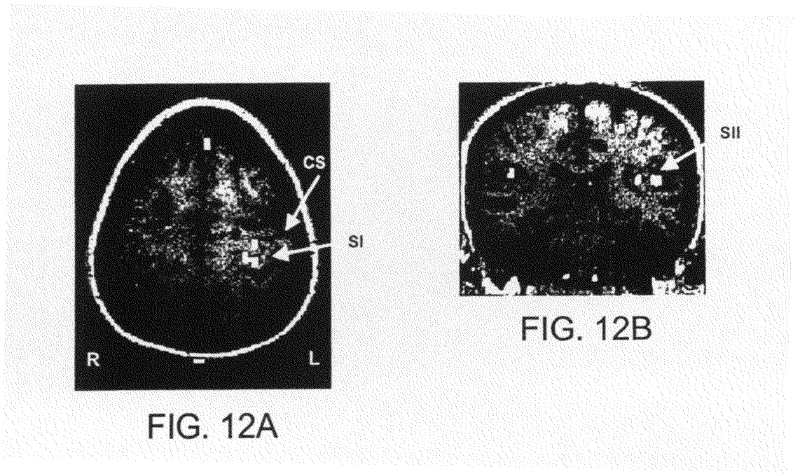

FIGS. 12A and 12B show representative activation maps of a subject produced by using the prototype MVD 100 to tap the thenar muscles (specifically abductor pollicis brevis and flexor pollicis brevis) of the right thumb. FIG. 12A illustrates activation in the contralateral primary (SI) somatosensory cortex, and FIG. 12B illustrates bilateral activation in the second (SII) somatosensory cortex. The location of the central sulcus (CS) is indicated with an arrow. These activation patterns have been observed to occur reliably in multiple subjects.

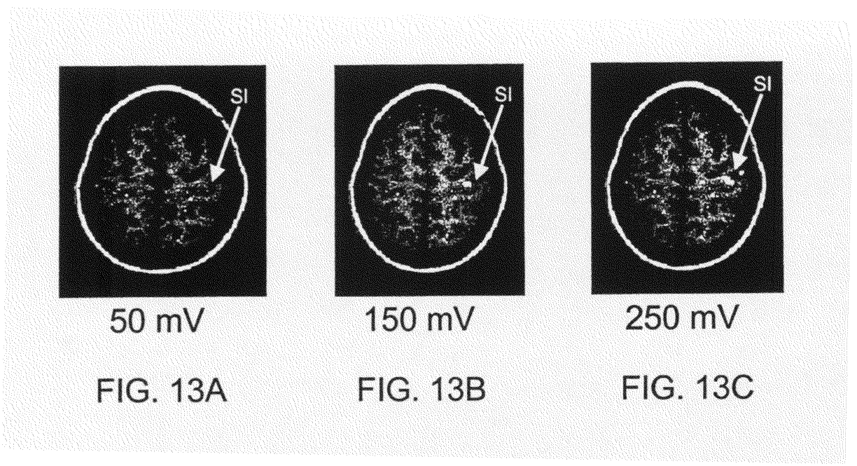

FIGS. 13A, 13B and 13C illustrate the effect of input voltage on SI activation for inputs of 50 mV, 150 mV, and 250 mV, respectively, in a representative subject examined using the prototype to tap the thenar muscles. Such an experiment in several volunteers would allow the relationship between input voltage and SI activation to be determined quantitatively to vibration. In this case, the lowest input voltage (FIG. 13A) appears to cause no activation despite the subject perceiving the stimulus, and the 250 mV input (FIG. 13C) appears to exhibit stronger activation than that produced using 150 mV (FIG. 13B). Lack of activation at the lowest stimulus amplitude is a reflection of the baseline signal-to-noise ratio on the 1.5 T scanner, and highlights the need to adjust stimulus amplitude to achieve reliable activation images in different subjects.

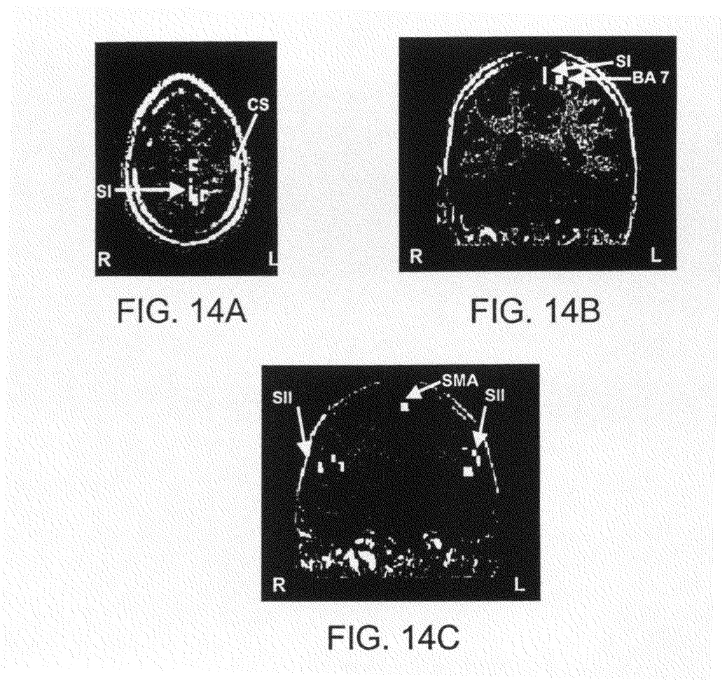

FIGS. 14A, 14B and 14C illustrate representative activation maps of one subject produced by using the MVD prototype 100 to tap the patellar tendon, strongly stimulating muscle spindles in the right quadriceps muscle and producing contralateral activation in the appropriate region of SI and parietal association cortex in Brodmann Area 7 (FIGS. 14A and 14B) as well as contralateral supplementary motor area (SMA) and bilateral SII (FIG. 14C). These results demonstrate the practicality of generating proprioceptive activation in the lower limb without directed movement or manipulation devices, and without producing significant stimulus-correlated head motion.

Devices utilizing Lorentz forces have been used previously in other aspects of MRI research, such as to generate compression and shear waves in MR elastography, and to control catheter tip deflection for interventional MRI applications. From the representative results shown in FIGS. 11A to 14C, it is clear that the MVDs of the present invention can be easily used to generate robust, reliable brain activations during fMRI. The properties of MVDs significantly augment other mechanisms used in fMRI to stimulate the somatosensory system: manual brushing, piezoelectric materials, air flow, and electrical stimulation of appropriate sensory afferents (eg the median nerve). Although manual brushing has been used widely for tactile stimulation, it is clear that MVDs and the other methods mentioned above that can be automated provide uniform stimulus delivery, or stimulus delivery at graded amplitudes. This reduces variability in time series data and improves the capability to map brain activation using correlation or fitting methods that make a priori assumptions about the idealized hemodynamic response produced by neuronal activation during fMRI experiments. Piezoelectric materials also produce highly reproducible vibration, but the vibration amplitude is too small (approximately microns) to provide practical coupling to transductive components for producing robust brushing or tapping stimuli. Without invasive placement of needle electrodes, electrical stimulation cannot directly mimic different types of natural somatosensory stimuli and separate the effects of different skin sensory receptors. Similarly, such separation would be difficult to achieve solely with the limited frequency response associated with controlled air flow.

As will be apparent to persons skilled in the art, various modifications and adaptations of the structure described above are possible without departure from the present invention, the scope of which is defined in the appended claims.

1.-27. (canceled)

28. A method of providing somatosensory stimuli to a subject when performing functional magnetic resonance imaging, said method comprising the steps of:
(a) providing a magnetomechanical vibrotactile device comprising a coil of wire;
(b) placing said device adjacent to an organ of a subject, wherein said organ is positioned within a magnetic field of a magnetic resonance imager;
(c) applying a time-varying current to said coil, wherein a force caused by an interaction of said magnetic field with said time-varying current produces vibratory motion of said device;
(d) coupling said vibratory motion to said organ to stimulate somatosensory receptors of said subject.

29. The method of claim 28, further comprising varying at least one of a temporal, frequency, and amplitude characteristics of said current.

30. The method of claim 28, wherein said characteristic of said current is varied to preferentially stimulate a specific class of somatosensory receptor.

31.-35. (canceled)

36. The method of claim 28, wherein said device further comprises a former, and wherein said coil is wound around said former.

37. The method according to claim 36 wherein said former is substantially cylindrical.

38. The method according to claim 36 wherein said former is substantially spherical.

39. The method according to claim 38 wherein said device further comprises two additional coils wound around said former, said first coil and said two additional coils being oriented in three orthogonal directions.

40. The method of claim 39, further comprising varying a current provided to at least one of the said three wire coils to achieve vibratory motion in a selected orientation.

41. The method of claim 28, further comprising indirectly coupling said vibratory motion to said organ using a coupling means, said coupling means comprising a proximal end and a distal end, wherein said proximal end is connected to said device, and said distal end comprises a contacting member adapted to couple said vibratory motion to said organ.

42. The method of claim 41, further comprising adapting said coupling means to convert rotational vibratory motion of said device into translational vibratory motion of said contacting member.

43. The method of claim 41, further comprising adapting said coupling means to convert rotational vibratory motion of said device into tapping vibratory motion of said contacting member.

44. The method of claim 41, wherein said contacting member further comprises a textured surface.

45. The method of claim 41, wherein said contacting member further comprises a brush.

46. The method of claim 28, wherein said organ is selected from the list comprising skin, bones, tendons, and eyes.

47. The method of claim 28 wherein said step of placing said device adjacent to an organ of said subject comprises integrating said device with an apparel article worn by said subject.

48. The method of claim 47 wherein said apparel article is a glove.

49. The method of claim 47 wherein said apparel article comprises an array of said devices.

50. A method of performing functional magnetic resonance imaging, said method comprising the steps of:
(a) providing a magnetomechanical vibrotactile device comprising a coil of wire;
(b) placing said device adjacent to an organ of a subject, wherein said organ is positioned within a magnetic field of a magnetic resonance imager;
(c) applying a time-varying current to said coil, wherein a force caused by an interaction of said magnetic field with said time-varying current produces vibratory motion of said device;
(d) coupling said vibratory motion to said organ to stimulate somatosensory receptors of said subject; and
(e) obtaining a magnetic resonance image.

51. The method of claim 50 wherein said vibratory motion is coupled to said organ while obtaining said magnetic resonance image.

52. The method of claim 50, wherein said device further comprises a former, and said coil is wound around said former.

53. The method of claim 50, further comprising indirectly coupling said vibratory motion to said organ using a coupling means, said coupling means comprising a proximal end and a distal end, wherein said proximal end is connected to said device, and said distal end comprises a contacting member adapted to couple said vibratory motion to said organ.

54. The method of claim 28, further comprising randomly varying at least one of a temporal, frequency, and amplitude characteristic of said current.
